(19) United States
(12) Patent Application Publication
(10) Pub. No.: US 2017/0348345 A1
(43) Pub. Date: Dec. 7, 2017

(54) Combination Therapy For Cancer

(71) Applicant: Gliotherapy Limited, Chipping Norton (GB)

(72) Inventors: Haritha SAMARANAYAKE, Kuopio (FI); Jere PIKKARAINEN, Kuopio (FI); Ann-Marie MAATTA, Kuopio (FI); Seppo YLA-HERTTUALA, Kuopio (FI)

(73) Assignee: Gliotherapy Limited

(21) Appl. No.: 15/644,860

(22) Filed: Jul. 10, 2017

Related U.S. Application Data
(63) Continuation of application No. 13/877,246, filed on Aug. 7, 2013.

(30) Foreign Application Priority Data
Jan. 18, 2011 (GB) GB1100804.2
Jan. 18, 2012 (GB) PCT/GB2012/050108

(51) Int. Cl. A61K 31/7088; A61K 48/00; A61K 45/06; A61K 38/45; A61K 31/522; C12N 9/12; A61K 31/495

(52) U.S. Cl. CPC A61K 31/7088; C12Y 207/01021; C12N 9/1211; A61K 48/0083; A61K 31/495; A61K 38/45; A61K 31/522; A61K 45/06

(57) ABSTRACT

A method of treating brain cancer in an immune-competent human patient by administering ternozolornide to the immune-competent human patient, the improvement comprising administering to said immune-competent human patient a viral vector.

### RELATED APPLICATIONS

This application is a continuation of U.S. Ser. No. 13/877246, tiled 1 Apr. 2013 and pending, which is the National Stage of PCT/GB2012/050108, tiled 18 Jan. 2012, which asserts priority to Great Britain application Serial No. 11.00804.2, filed 18 Jan. 2011. The contents of the foregoing are here incorporated by reference.

### FIELD OF INVENTION

This invention relates to a drug combination for the treatment of cancer or of a disease characterised by an impaired mismatch repair (MMR) pathway.

### BACKGROUND OF THE INVENTION

Herpes simplex virus type 1 thymidine kinase (HSV-tk) gene therapy is based on the prodrug activating enzyme that converts a non-toxic compounds such as ganciclovir, (GCV) into a toxic metabolite. The cell destruction by HSV-tk/GCV is cell cycle dependent, where only dividing cells will be affected. This is of particular advantage in brain cancer gene therapy, where the rapidly dividing tumour cells are surrounded by non-dividing normal brain cells. Therapy by HSV-tk is disclosed in EP1 135513.

Temozolomide (TMZ, imidazole tetrazinone) is an oral alkylating agent that can cross the blood brain barrier (BBB). Temozolomide is an oral alkylating agent that is a derivative of dacarbazine. TMZ undergoes spontaneous hydrolysis at physiological pH to its active form 3-methyl-(triazen-1-yl) imidazole-4 carboxyamide (MTIC). The primary mode of cytotoxicity is by adding a methyl group at Opposition of guanine (O⁶-mG).

O⁶-mG by itself is not toxic to the cells. However, O⁶-mGs will become cytotoxic as a result of repeated cycles of futile efforts at repair by mismatch repair (MMR) pathway. This will ultimately lead to DNA strand breaks. It is known that a functional MMR pathway is essential to make cells sensitive to TMZ, in the absence of an active MGMT repair pathway (which occurs in 50% of malignant gliomas). Furthermore, defects in the MMR pathway can contribute to almost 100-fold resistance to alkylating agents such as TMZ.

A paper by Rainov et al (Cancer Gene Therapy, Vol 8, No 9, 2001: pp 662-668), reports some experiments on the combination of HSV-tk/GCV gene therapy and TMZ chemotherapy, but the data do not show any compelling evidence of synergy.

### SUMMARY OF THE INVENTION

The present invention is based on the discovery that HSV-tk gene therapy increases the gene expression of key mismatch repair (MMR) pathway proteins, namely MSH2 and MLH1. This led to the finding that HSV-tk/GCV gene therapy sensitises cells to chemotherapeutic agents, such as temozolomide (TMZ).

A study designed by the inventors confirmed that a combination of vector/prodrug gene therapy (such as HSV-tk/GCV) and a cytotoxic agent, has much improved efficacy in certain diseases (cancer was tested, but it is believed that this applies to all diseases characterised by an impaired MMR pathway), when compared to the use of either of the components alone, i.e. chemotherapy or vector/prodrug gene therapy.

It was also found that the administration protocol of these components is key to the surprising technical effect observed in the invention, i.e. the synergy. The inventors have found that the upregulation of the MMR pathway by vector/prodrug gene therapy takes approximately 2 days, and lasts for a maximum of 7 days after stopping prodrug therapy. Therefore, in order to see synergy it is necessary to begin administering the cytotoxic agent no later than 7 days after finishing prodrug therapy.

Furthermore, when the condition to be treated is characterised by an impaired MMR pathway, it is believed that a therapeutic benefit may be achieved by administering only the vector/prodrug gene therapy.

In a first aspect, the present invention is characterised by a new dosage regimen. Therefore, according to a first aspect, the present invention is an agent comprising a vector having a functional gene, a prodrug which can be converted into a cytotoxic agent by an expression product of the gene, and another cytotoxic agent, as a combined preparation for simultaneous, sequential or separate use in the therapy of cancer or of a disease characterised by an impaired mismatch repair (MMR) pathway, wherein the dosage regimen comprises beginning prodrug therapy after the vector has been administered, and beginning the another cytotoxic agent therapy no later than 7 days after the prodrug therapy has finished.

According to a second aspect, the present invention is an agent comprising a vector having a functional gene, and a prodrug which can be converted into a cytotoxic agent by an expression product of the gene, as a combined preparation for simultaneous, sequential or separate use in the therapy of a disease characterised by an impaired mismatch repair (MMR) pathway.

According to a third aspect, a method of treating glioblastoma multiforme, comprises the steps of:
a. Diagnosing in a human patient glioblastoma multiforme;
b. Identifying in said patient at least one glioblastoma multiforme tumor;
c. Resectioning said glioblastoma multiforme tumor to remove at least part of said glioblastoma multiforme tumor and expose tumor bed tissue; d. Administering to said tumor bed tissue an AdHSV-i/c adenoviral vector having a gene coding for thymidine kinase, whereby said AdHSV-i/c adenoviral vector transfects said tumor bed tissue and said tumor bed tissue expresses said gene coding for thymidine kinase;
e. Within about 5 to about 19 days after administering said adenoviral vector to said human patient, further administering to said human patient ganciclovir;
f. Administering to said human patient temozolomide per os or by intravenous infusion.

### DESCRIPTION OF THE FIGURES

FIGS. 1 and 2 show mean tumour volume at days 28 and 42 for different HSV-tk/GCV and TMZ/dosage regimens.

FIG. 3 shows survival rate for different HSV-tk/GCV and TMZ dosage regimens.

### DESCRIPTION OF THE PREFERRED EMBODIMENTS

The present invention requires the administration of a vector having a functional gene, and a prodrug which can be converted by an expression product of that gene, into a cytotoxic agent. Preferably, the functional gene is a functional thymidine kinase gene. Preferably, the prodrug is ganciclovir or its analogues. It will be understood that the prodrug therapy should commence after the vector has been administered. Preferably the prodrug is administered from 5 to 19 days after administration of the vector.

Alternatively, suicide genes such as cytosine deminase, cytochrome P450, E coli purine nucleoside phosphorylase and carboxypeptidase G2, are suitable for use in the invention. Those suicide genes can be used in combination with suitable prodrugs, such as 5-fluorocytosine, cyclophosphamide, 6-methylepurine or F-araAMP or 4-benzoyl-L-glutamic acid (CMDA) or their chemical analogs, respectively. In one embodiment, the suicide gene, i.e. the vector, is cytosine deminase, and the prodrug is 5-fluorocytosine is suitable for use in the invention.

The vector is preferably locally administrated. When the therapy is of a cancerous tumour, for example, the vector may be administered directly into that cancerous tumour. Alternatively, it may be preferable to surgically remove the cancerous tumour, and then administer the vector into the wall of the tumour cavity.

As used herein, the term “wall of the tumour cavity” means the area of apparently healthy tissue (i.e. tissue which is apparently healthy to the eye of a surgeon) that remains once a tumour (or part of that tumour) is removed. Although the tissue is apparently healthy, it may contain malignant cells. The term “wall of the tumour cavity” refers to an area of non-tumour mass.

Preferably, the tumour resection is complete as possible, i.e. more than 90%, 95% or 98%. In a preferred embodiment, the vector is administered by injection approximately 1 cm (preferably between 0.5 cm and 5 cm, more preferably between 0.8 cm and 3 cm) deep into the wall of the tumour cavity. This ensures that the vector is into healthy tissue, i.e. is targeting primarily healthy cells (although it is appreciated that some malignant cells may reside in that area of apparently healthy tissue).

The vector that is used to transfer the gene may be any viral vector. However, it is preferred that it is derived from an adenovirus or a lentivirus. More preferably, it is derived from adenovirus.

The present invention is a combination therapy, comprising the administration of a gene therapy vector, a prodrug and a cytotoxic agent. The cytotoxic agent is preferably different from the cytotoxic agent that results from conversion of the prodrug (for example conversion of the ganciclovir), but otherwise the exact nature of the cytotoxic agent is not crucial, but it should preferably be a drug whose function is impaired by impaired MMR pathway. Some preferred cytotoxic agents are:
a) a chloroethylating agents such as carmustine, lomustine, fotemustine, nimustine, ranimustine or streptozocin;
b) a non-classical alkylating agent such as procarbazine;
c) a methylating triazine such as temozolomide, dacarbazine, altretamine, or mitobronitol;
d) a DNA cross-linking agent such as cisplatin, carboplatin, nedaplatin, oxaliplatin, triplatin, tetranitrate or satraplatin;
e) a topoisomerase II inhibitor such as doxorubicin, epirubicin, aclarubicin, daunorubicin, idarubicin, amrubicin, pirarubicin, valrubicin or zorubicin, mitoxantrone or pixantrone;
f) a topoisomerase I inhibitor such as topotecan, camptothesin, irinotecan, rubitecan or belotecan;
g) an anti metabolite (pyrmidine analogue) such as 5-FU, capecitabine, tegafur, carmofur, floxuridine or cytarabine;
h) an anti metabolite (purine analogue) such as 6-thioguanine or mercaptopurine; or
i) a cytotoxic DNA alkylating agent.

The most preferred cytotoxic agent is temozolomide (TMZ).

For synergy between vector/prod rug/cytotoxic, it is necessary for the MMR pathway to become upregulated, and therefore administration protocol/dosage regimen is key.

As used herein, “cytotoxic therapy” and “prodrug therapy” means the cytotoxic and prodrug dosage regimens, courses of treatment. Those therapies are for a specified period of time. The vector, however, need only be administered once.

Preferably, the another cytotoxic agent therapy begins no later than 7 days after prodrug therapy has finished. More preferably, the cytotoxic agent therapy begins no later than 6, 5, 4, 3, 2 or 1 day after prodrug therapy has finished. Preferably, the cytotoxic agent therapy begins less than 1 day after prodrug therapy finishes.

For the avoidance of doubt, included within the scope of the invention is both the situation where cytotoxic therapy is started immediately after prodrug therapy has finished, and also the situation where cytotoxic therapy is started before the prodrug therapy has finished (i.e. there is a period of simultaneous administration.

The cytotoxic therapy and the prodrug therapy may be started at the same time. Although, preferably, the cytotoxic agent therapy begins no earlier than 2 days after prodrug therapy begins. This allows for most efficient administration as the cytotoxic and prodrug are only combined once the MMR pathway has been upregulated. This is the most efficient dosage regimen.

Preferably, the prodrug therapy and the another cytotoxic agent therapy overlaps. More preferably, the therapies overlap for at least 3 days. More preferably, they overlap for at least 7, 10, 14 or 18 days.

Preferably, the prodrug therapy lasts for from 10 to 20 days. More preferably, it lasts for from 11 to 19, 12 to 18 or 13 to 17 days. Preferably, it lasts for 14 days.

In a preferred embodiment, the prodrug therapy begins from 2 to 5 days after vector administration (gene transfer). More preferably, the prodrug therapy begins at 5 days after gene transfer.

Preferably, the another cytotoxic therapy should begin at the earliest at 2 days after starting prodrug therapy, and at the latest at 7 days after stopping prodrug therapy.

The another cytotoxic agent therapy should begin no earlier than simultaneously with the commencement of prodrug therapy. it will be appreciated that it is preferred for the another cytotoxic agent therapy to begin no earlier than 2 days after commencement of prodrug therapy.

The upregulation of the MMR pathway is key to the invention. Therefore, it will be appreciated that the agent of the invention is useful in the treatment of a number of conditions. Examples of those conditions are cancer, actinic keratosis, pterygium diabetic retinopathy, atherosclerosis, asthma, chronic obstructive pulmonary disease, sarcoidosis, idiopathic pulmonary fibrosis, rheumatoid arthritis, pseudoexfoliation syndrome of the eye and Alzheimer's disease.

The most preferred therapy is of cancer. Preferably, the therapy is of a cancerous tumour, such as malignant glioma, or a tumour of the prostate. An agent of the invention may be used in the therapy of a cancer characterised by a normal or an impaired MMR pathway.

In a further preferred embodiment, an agent according to the present invention, when used to treat a cancerous tumour, also includes the administration of radiation. The radiation is preferably administered after the administration of the vector and the prodrug, and radiation therapy preferably starts at the same time as the cytotoxic chemotherapeutic agent (preferably, therapy is simultaneous).

The following study illustrates the present invention.

Study

A study was conducted concerning tumour growth rate in a rat glioma model. There were 6 patient groups. Details of agents administered and the dosage regimen are shown in Table 1 below.

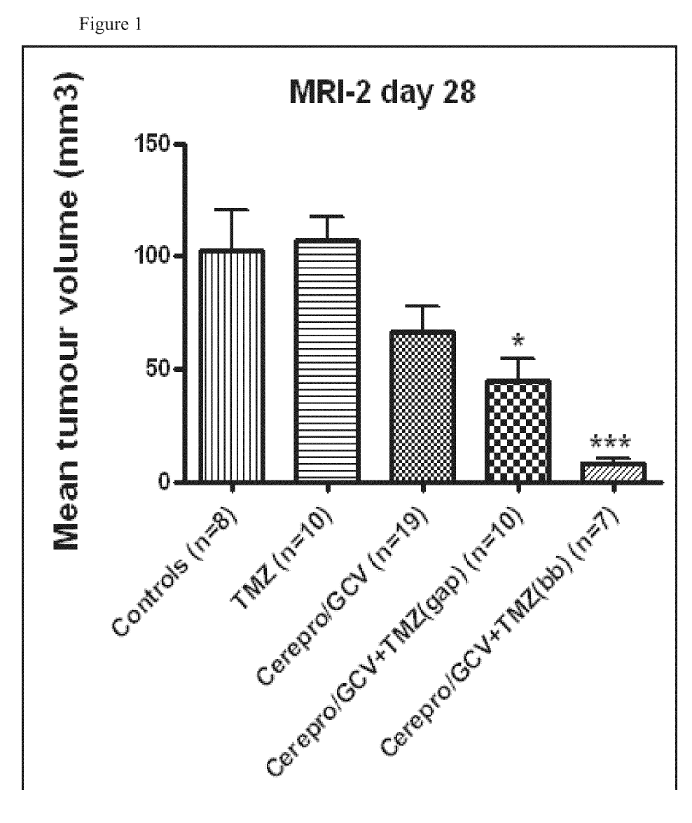

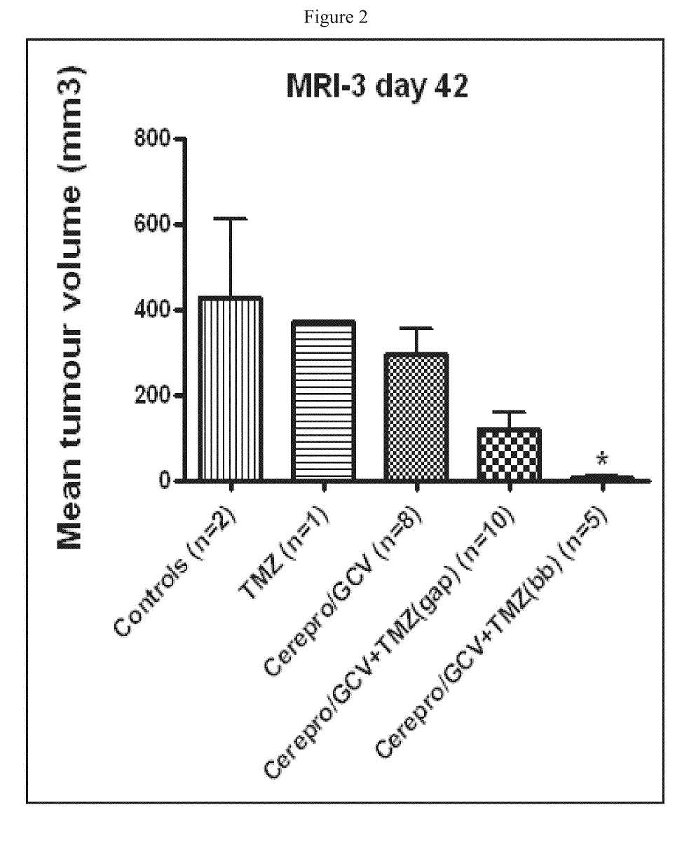

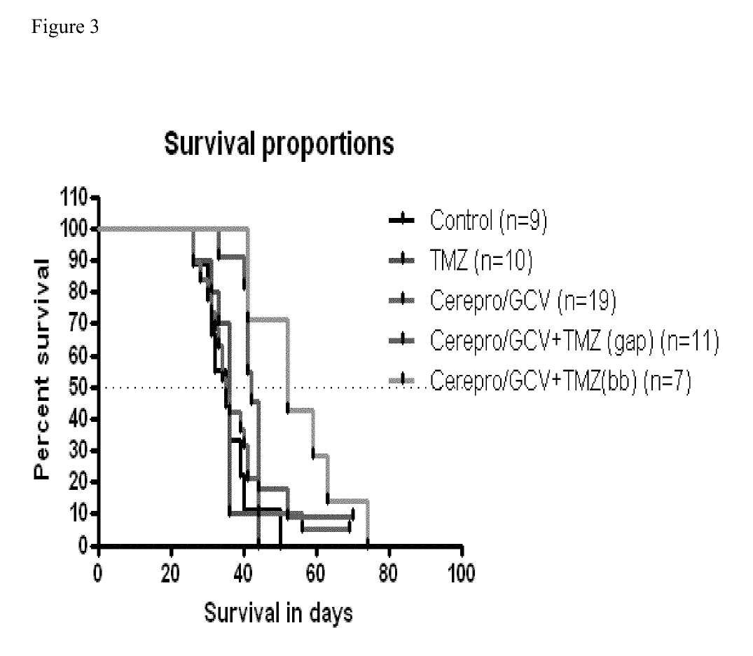

The results are shown in FIGS. 1 and 2. Group 5 shows the biggest decrease in tumour size.

A second study was conducted in the rat glioma model concerning survival rates. The data (FIG. 2) show that Group 6 had the longest survival rate, closely followed by group 5. This partly led the inventors to devise the dosage regimen of the invention (as slight overlap of prodrug/cytotoxic therapy is beneficial).

1. A method of treating brain cancer in an immunocompetent human patient comprising: administering to said immunocompetent human patient an anti-cancer effective amount of at least one chemotherapeutic, and administering to said immunocompetent human patient an anti-cancer effective amount of a viral vector.

2. The method of claim 1, wherein said viral vector is administered in an amount of about 1 to 3×10³ cfu.

3. The method of claim 1, further comprising:
resecting at least part of said brain cancer.

4. The method of claim 3, wherein said resecting forms a cavity and wherein said cavity has a cavity wall, and wherein said administration of said viral vector comprises administration to the wall of the cavity formed by the resecting.

5. The method of claim 1, wherein said viral vector comprises virus selected from the group consisting of: lentivirus and adenovirus.

6. The method of claim 1, wherein said viral vector is replication competent.

7. The method of claim 1, wherein said viral vector only infects dividing human cells.

8. The method of claim 6, wherein said viral vector only infects dividing human cells.

9. The method of claim 1, wherein said viral vector comprises a thymidine kinase transgene, and wherein said method of treatment further comprises administering to said human patient ganciclovir or an analogue thereof.

10. The method of claim 1, wherein said brain cancer is selected from the group consisting of: malignant glioma, glioblastoma multiforme and anaplastic astrocytoma.

11. The method of claim 1, further comprising administering to said human patient radiotherapy.

12. The method of claim 1, wherein said administration of said temozolomide lasts for not more than about 50 days.

13. A method of treating brain cancer in a human patient comprising: (a) administering to said human patient an anti-cancer effective amount of at least one chemotherapeutic, and (b) resecting at least part of said brain cancer to form a tumor cavity having a cavity wall and then administering to said cavity wall an anti-cancer effective amount of a viral vector.

14. The method of claim 13, wherein said viral vector comprises virus selected from the group consisting of: lentivirus and adenovirus.

15. The method of claim 13, wherein said viral vector is replication competent.

16. The method of claim 13, wherein said viral vector only infects dividing human cells.

17. The method of claim 15, wherein said viral vector only infects dividing human cells.

18. The method of claim 13, wherein said viral vector comprises a thymidine kinase transgene, and wherein said method of treatment further comprises administering to said human patient ganciclovir or an analogue thereof.

19. The method of claim 13, wherein said brain cancer is selected from the group consisting of: malignant glioma, glioblastoma multiforme and anaplastic astrocytoma.

20. The method of claim 1, wherein said chemotherapeutic comprises an alkylating agent.

21. The method of claim 20, wherein said alkylating agent comprises temozolomide.

22. The method of claim 13, wherein said chemotherapeutic comprises an alkylating agent.

23. The method of claim 22, wherein said alkylating agent comprises temozolomide.

24. A method of treating brain cancer in a human patient comprising: (a) resecting at least part of said brain cancer to form a. tumor cavity having a cavity wall and then (b) administering to said human patient viral vector in an amount adequate to induce the expression of interferon.
